(19) United States
(12) Patent Application Publication
(10) Pub. No.: US 2022/0249585 A1
(43) Pub. Date: Aug. 11, 2022

(54) MEDICATION COATED WITH HEMP OR OTHER CANNABINOID

(71) Applicant: KHUBANI, Ajit, Fairfield, NJ (US)

(72) Inventor: Ajit Khubani, Fairfield, NJ (US)

(73) Assignee: Hempvana, LLC, Miami Beach, FL (US)

(21) Appl. No.: 17/629,760

(22) PCT Filed: Jul. 25, 2020
(86) PCT No.: PCT/US2020/043638

§ 371 Date: Jan. 24, 2022

Related U.S. Application Data
(60) Provisional application No. 62/878,707, filed on Jul. 25, 2019.

(51) Int. Cl. A61K 36/185; A61K 9/00; A61K 9/16; A61K 31/05; A61K 31/355; A61K 31/167; A61K 31/60; A61K 31/192

(52) U.S. Cl. CPC A61K 36/185; A61K 9/0056; A61K 9/167; A61K 31/192; A61K 31/355; A61K 31/167; A61K 31/60; A61K 31/05

(57) ABSTRACT

A medication comprising a medication coated with hemp, or a composition including hemp oil, hemp seed oil, or another cannabinoid is provided.

### CROSS-REFERENCE TO RELATED APPLICATIONS

This application claims the benefit of U.S. Provisional Application No. 62/878,707 filed Jul. 25, 2019.

### TECHNICAL FIELD

The present invention relates to medication coated with hemp or a composition including hemp oil or hemp seed oil or another cannabinoid.

### BACKGROUND OF THE INVENTION

Medications are essential to a healthy life. There are many ways of administering medication. One method is delivering the medication orally, in the form of a tablet, a capsule, a gel capsule, a soft gel a liquid capsule, a gummy bear, a caplet, an orally disintegrating tablet, a pill, a lozenge, a pastille, a pellet, etc. In order to effectively administer medications orally, the medication is often coated to achieve a particular purpose.

Accordingly, there is a need for alternative coating compositions for effective administering of medication. There is also a need for coating compositions with improved efficacy and benefits. The objective of this application is to provide alternative and improved medication coated with cannabinoids and methods for coating medication with a cannabinoid composition.

### SUMMARY OF THE INVENTION

The subject invention provides a medication comprising a medication coated with hemp, or a composition including hemp oil, hemp seed oil, or another cannabinoid.

In an embodiment of this disclosure, there is provided a medication coated with hemp or a material including hemp oil or hemp seed oil or another cannabinoid, in an effective amount of the cannabinoid.

In another embodiment, a medication is coated with a cannabinoid (CBD) oil or hemp extract, in an effective amount of the CBD oil or hemp extract.

In another embodiment, a medication comprises an analgesic coated with an effective amount of cannabinoid on the exterior of the analgesic.

In another embodiment, a medication comprises over-the-counter (OTC) medication coated with an effective amount of cannabinoid on the exterior of the OTC medication (also reference herein as “cannabinoid-coated medication”.

In another embodiment, a medication comprises a vitamin supplement coated with an effective amount of cannabinoid on the exterior of the vitamin supplement.

The aforementioned medication can be in the form of a tablet, a capsule, a gel capsule, a soft gel a liquid capsule, a gummy bear, a caplet, an orally disintegrating tablet, a pill, a lozenge, a pastille, a pellet, etc.

The aforementioned medication is not limited to vitamin supplements and analgesics, and can include other OTC medicine.

Various other inventive aspects can be employed integrated or otherwise included, as discussed infra.

### DETAILED DESCRIPTION OF THE INVENTION

The following detailed description of embodiments and examples are set forth to aid in an understanding of the subject matter of this disclosure but are not intended to, and should not be construed to, limit in any way the disclosure as a whole.

A medication coated with hemp, hemp oil, hemp seed oil or another a cannabinoid, in an effective amount, may be an efficacious means of delivering such medication to a subject.

For example, such medication can be constituted to provide chronic pain relief by impacting endocannabinoid receptor activity, reducing inflammation and interacting with neurotransmitters. Additionally, such medication may provide a relief to both depression and anxiety by its ability to act on the brain's receptors for serotonin, a neurotransmitter that regulates mood and social behavior.

There may be synergistic advantages of employing such a medication to obtain pain relief achieved at lowered concentrations of both analgesic and cannabinoid and providing a prolonged relief of pain. Such medication may be capable of achieving unique product performance objectives not otherwise achievable by conventional tablets. In addition, the medication may have advantages, including control of the delivery rate of single or two or more different active ingredients, while at the same time providing separation of incompatible active ingredients from one another or controlled release of active ingredients from one layer through utilization of the functional property of adjacent layer. Moreover, such medication may include variation of the total surface area available for the active ingredient layer, either by sandwiching with one or two inactive layers in order to achieve swellable/erodible barriers for delayed release.

In one embodiment, a novel cannabinoid-coated medication comprises a medication coated with an effective amount of cannabinoid on the exterior of the medication, partially or in its entirety. The medication can be in the form of a tablet, capsule, gel capsule, liquid capsule, gummy bear, caplet, orally disintegrating tablet, pill, lozenge, pastille or pellet.

In another aspect of this embodiment, a novel cannabinoid-coated medication comprises a medication coated with an effective amount of cannabinoid covering the exterior of the medication partially or in its entirety, The medication can be in the form of a tablet, capsule, gel capsule, liquid capsule, gummy bear, caplet, orally disintegrating tablet, pill, lozenge, pastille or pellet.

In yet another aspect of this embodiment, a novel cannabinoid-coated medication comprises a medication blended with a cannabinoid and coated with an effective amount of cannabinoid covering the exterior of the medication blended with cannabinoid partially or in its entirety. The medication can be in the form of a tablet, capsule, gel capsule, soft gel liquid capsule, gummy bear, caplet, orally disintegrating tablet, pill, lozenge, pastille or pellet.

In another embodiment, a cannabinoid-coated medication comprising a medication coated with a dronabinol powder, in an effective amount, may be a valuable means of delivering such medication to a subject.

In one aspect of this embodiment, a novel cannabinoid-coated medication comprises a medication coated with an effective amount of dronabinol powder covering the exterior of the medication partially or in its entirety. The medication can be in the form of a tablet, capsule, gel capsule, liquid capsule, gummy bear, caplet, orally disintegrating tablet, pill, lozenge, pastille or pellet.

In yet another aspect of this embodiment, a novel cannabinoid-coated medication comprises a medication blended with a dronabinol powder and coated with an effective amount of dronabinol powder covering the exterior of the medication blended with dronabinol powder partially or in its entirety. The medication can be in the form of a tablet, capsule, gel capsule, soft gel liquid capsule, gummy bear, caplet, orally disintegrating tablet, pill, lozenge, pastille or pellet.

Dronabinol, which contains THC, can provide longer-lasting pain relief when employed in a cannabinoid-coated medication.

In another embodiment, a novel cannabinoid-coated medication comprises a medication coated with an effective amount of cannabinoid resin covering the exterior of the medication partially or in its entirety. The medication can be in the form of a tablet, capsule, gel capsule, liquid capsule, gummy bear, caplet, orally disintegrating tablet, pill, lozenge, pastille or pellet.

In yet another aspect of this embodiment, a novel cannabinoid-coated medication comprises a medication blended with the cannabinoid resin and coated with an effective amount of cannabinoid resin covering the exterior of the medication blended with cannabinoid resin partially or in its entirety. The medication can be in the form of a tablet, capsule, gel capsule, soft gel liquid capsule, gummy bear, caplet, orally disintegrating tablet, pill, lozenge, pastille or pellet.

In another embodiment, a cannabinoid-coated medication comprises a medication coated with a hemp oil, in an effective amount, may be an efficacious means of delivering such medication to a subject.

A cannabinoid-coated medication comprising a medication coated with hemp oil can provide advantages. For example, hemp oil is derived from hemp seed or from Cannabis sativa, which an extremely fast-growing crop, making it an environmentally friendly crop. Additionally, hemp oil is highly nutritious and helpful for the skin and contains less than 0.3 percent THC.

In one aspect of this embodiment, a novel cannabinoid-coated medication comprises a medication coated with an effective amount of hemp oil, on the exterior of the medication, partially or in its entirety. The medication can be in the form of a tablet, capsule, gel capsule, liquid capsule, gummy bear, caplet, orally disintegrating tablet, pill, lozenge, pastille, pellet, etc.

In another aspect of this embodiment, a novel cannabinoid-coated medication comprises a medication coated with an effective amount of hemp oil covering the exterior of the medication partially or in its entirety. The medication can be in the form of a tablet, capsule, gel capsule, liquid capsule, gummy bear, caplet, orally disintegrating tablet, pill, lozenge, pastille or pellet.

In yet another aspect of this embodiment, a novel cannabinoid-coated medication comprises a medication blended with the hemp oil, and coated with an effective amount of hemp oil covering the exterior of the medication blended with hemp oil. The medication can be in the form of a tablet, capsule, gel capsule, soft gel liquid capsule, gummy bear, caplet, orally disintegrating tablet, pill, lozenge, pastille, pellet, etc.

In another embodiment, a novel cannabinoid-coated medication comprises a medication coated with an effective amount of cannabis seed extract on the exterior of the medication partially or in its entirety. The medication can be in the form of a tablet, capsule, gel capsule, liquid capsule, gummy bear, caplet, orally disintegrating tablet, pill, lozenge, pastille, pellet, etc.

In another aspect of this embodiment, a novel cannabinoid-coated medication comprises a medication coated with an effective amount of cannabis seed extract covering the exterior of the medication partially or in its entirety. The medication can be in the form of a tablet, capsule, gel capsule, liquid capsule, gummy bear, caplet, orally disintegrating tablet, pill, lozenge, pastille, pellet, etc.

In yet another aspect of this embodiment, a novel cannabinoid-coated medication comprises a medication blended with cannabis seed extract, and coated with an effective amount of cannabis seed extract covering the exterior of the medication blended with cannabis seed extract partially or in its entirety. The medication can be in the form of a tablet, capsule, gel capsule, soft gel liquid capsule, gummy bear, caplet, orally disintegrating tablet, pill, lozenge, pastille, pellet, etc.

In another embodiment, a novel cannabinoid-coated medication comprises a medication coated with an effective amount of CBD oil or hemp extract on the exterior of the medication partially or in its entirety. The medication can be in the form of a tablet, capsule, gel capsule, liquid capsule, gummy bear, caplet, orally disintegrating tablet, pill, lozenge, pastille, pellet, etc.

Another aspect of this embodiment, a novel cannabinoid-coated medication comprises a medication coated with an effective amount of CBD oil or hemp extract covering the exterior of the medication partially or in its entirety. The medication can be in the form of a tablet, capsule, gel capsule, liquid capsule, gummy bear, caplet, orally disintegrating tablet, pill, lozenge, pastille, pellet, etc.

In yet another aspect of this embodiment, a novel cannabinoid-coated medication comprises a medication blended with the CBD oil or hemp extract, and coated with an effective amount of CBD oil or hemp extract covering the exterior of the medication blended with CBD oil or hemp extract partially or in its entirety. The medication can be in the form of a tablet, capsule, gel capsule, soft gel liquid capsule, gummy bear, caplet, orally disintegrating tablet, pill, lozenge, pastille, pellet, etc.

In another embodiment, a novel cannabinoid-coated medication comprises a medication coated with an effective amount of one or more a cannabinoid, dronabinol powder, cannabinoid resin, cannabis seed extract, hemp oil, CBD oil or hemp extract or a combination thereof on the exterior of the medication, partially or in its entirety. The medication can be in the form of a tablet, capsule, gel capsule, liquid capsule, gummy bear, caplet, orally disintegrating tablet, pill, lozenge, pastille or pellet.

In another embodiment, a novel cannabinoid-coated medication comprises a medication blended with one or more of following, a cannabinoid, dronabinol powder, cannabinoid resin, cannabis seed extract, hemp oil, CBD oil or hemp extract or a combination thereof and being coated with an effective amount of one or more of the following a cannabinoid, dronabinol powder, cannabinoid resin, cannabis seed extract, hemp oil, CBD oil or hemp extract or a combination thereof on the exterior of the medication, partially or in its entirety. The medication can be in the form of a tablet, capsule, gel capsule, liquid capsule, gummy bear, caplet, orally disintegrating tablet, pill, lozenge, pastille or pellet.

In one aspect of this embodiment, the cannabinoid blended with the medication is the same cannabinoid coated on the exterior of the medication. In another aspect of this embodiment, the cannabinoid blended with the medication is different than the cannabinoid coated on the exterior of the medication.

For example, in one aspect a novel cannabinoid-coated medication comprises a medication blended with the CBD oil or hemp extract, and coated with an effective amount of CBD oil or hemp extract covering the exterior of the blended medication.

In another example, a novel cannabinoid-coated medication comprises a medication blended with the hemp oil, and coated with an effective amount of CBD oil covering the exterior of the blended medication.

In another embodiment, a novel cannabinoid-coated medication comprises a medication that is coated with a coating composition. Such a coating composting can include one or more cannabinoids compounds, and one or more additional compounds.

For example, medication, in an embodiment of this disclosure, can be formed by coating a medication comprising a coating composition comprising cannabinoid and optionally one or more of the following: water, mineral oil, glyceryl stearate SE, cetyl alcohol, glycerin, isopropyl palmitate, sunflower (helianthus annuus) seed oil, phenoxyethanol, ethylhexylglycerin, bergamot (citrus aurantium bergamia) fruit oil, tocopheryl acetate, disodium EDTA aloe vera (aloe barbadensis) leaf extract, marigold (calendula officinalis) flower extract, retinyl palmitate, and cucumber(cucumis sativus) fruit extract.

As another example, the cannabinoid-coated medication can be formed by coating a medication with a coating composition comprising cannabinoid and optionally one or more of the following: cocos nucifera (coconut) oil, hydrogenated soybean oil, ozokerite, beeswax, carthamus tinctorius (safflower) seed, oil melia azadirachta seed oil, copernicia cerifera (carnauba) wax, nigella sativa seed oil, pongamia glabra seed oil, dimethicone, melaleuca viridiflora leaf oil, tocopheryl acetate, isopropyl myristate, caprylyl glycol, phenoxyethanol, melaleuca leucadendron cajaput oil, theobroma cacao (cocoa) seed butter, butyrospermum parkii (shea) butter, sorbitan oleate, cymbopogon schoenanthus oil, cymbopogon martini oil, citrus aurantium amara (bitter orange) peel oil, cedrus atlantica bark oil, rosmarinus officinalis (rosemary) leaf oil, melaleuca alternifolia (tea tree) leaf oil, eucalyptus globulus leaf oil, prunus amygdalus dulcis (sweet almond) oil, olea europaea (olive) fruit oil, persea gratissima (avocado) oil, helianthus annuus (sunflower) seed oil, oryza sativa (rice) bran oil, salvia sclarea (clary) oil, canarium commune gum oil and retinyl palm itate.

As another example, the cannabinoid-coated medication can be formed by coating a medication with a coating composition comprising cannabinoid and optionally one or more of the following: trolamine salicylate, water/aqua, ethyl hexyl stearate, butylene glycol, dimethicone, stearic acid, caprlic/capric triglyceride, glyceryl stearate, PEG-100 stearate, cetostearyl alcohol, sunflower oil, allantoin, tocopheryl acetate, ethylhexylglycerin, isohexadecane, sodium acrylate/sodium acryloyldimethyl taurate copolymer, acrylates/C10-30 alkyl acrylate crosspolymer, caprylyl glycol, hexylene glycol, polysorbate 80, disodium EDTA, D&C Green No. 5 (CI 61570), sodium hydroxide and phenoxyethanol.

As another example, the cannabinoid-coated medication can be formed by coating a medication with a coating composition comprising cannabinoid and optionally one or more of the following: menthol United States Pharmacopeia (USP), arctium lappa root extract, aloe barbadensis leaf extract, arnica montana flower extract, Boswellia carteril resin extract, calendula officinalis extract, camellia sinensis leaf extract, camphor, carbomer, FD&C Blue No. 1, FD&C Yellow No. 5, glycerin, Hex paragiariensis leaf extract, isopropyl alcohol, isopropyl myristate, Melissa officinalis (lemon balm) extract, silica, tocopheryl acetate, triethanolamine, and water, wherein the cannabinoid-coated medication comprises 8% by weight of menthol USP.

The medication can include an analgesic, over the counter (OTC) medication, and/or vitamin supplements. In particular, in one aspect the medication is an analgesic, which is selected from one or more of the following: a naproxen sodium, ibuprofen, acetaminophen, antihistamines,

In one aspect the medication is an Over the Counter (OTC) medication. In yet another aspect, the medication is a vitamin supplement which is selected from one or more of the following: multivitamin, dietary supplement with vitamins, dietary minerals, and other nutritional elements.

In another exemplary embodiment, the cannabinoid-coated medication comprising a medication coated with a cannabinoid, includes customized tablet having a plurality of layers, with the layers including at least two layers including: an instantaneous release layer and a delayed release layer. The delayed release layer may comprise a cannabinoid (e.g., dronabinol powder, cannabinoid resin), cannabis seed extract, hemp oil, CBD Oil or Hemp Extract or a combination thereof.

In another aspect of this embodiment, the cannabinoid-coated medication comprising a medication coated with a cannabinoid, includes the delayed release layer can be achieved with an enteric coating. The enteric coating on the cannabinoid-coated medication may be either of: a single layer tablet, where the active agents are administered prior to compression into a tablet form, or a plurality of layers, such as a two or three layer tablet, wherein each of an active agent or combinations of active agents are present in a separate layer within the compressed tablet form.

In another aspect of this embodiment, a dispersion of the delayed release layer can be homogeneous, or heterogeneous.

In yet another aspect of this embodiment, the dispersion of the instantaneous release layer can be homogeneous, or heterogeneous.

According to another exemplary embodiment, the cannabinoid-coated medication comprising a medication coated with a cannabinoid, includes a customized tablet comprised of a plurality of layers, with the layers including at least two layers including: a first layer comprising an analgesics which provides pain relief, and a second layer comprising cannabinoid which acts as a coating in the cannabinoid-coated medication.

In another aspect of this exemplary embodiment, the first layer can optionally include one or more of the following: an over the counter medication, an analgesic, a vitamin supplement.

In another aspect of this exemplary embodiment, the second layer, can optionally include one or more of the following: a cannabinoid, dronabinol powder, cannabinoid resin, cannabis seed extract, hemp oil, CBD Oil or Hemp Extract or a combination thereof.

In yet another aspect of this exemplary embodiment, the second layer of the cannabinoid-coated medication can be an outer layer of the cannabinoid-coated medication covering the first layer.

In another aspect of this exemplary embodiment, the dispersion of the first layer can be homogeneous, or heterogeneous.

In another aspect of this exemplary embodiment, the dispersion of the second layer can be homogeneous, or heterogeneous.

According to another exemplary embodiment, a cannabinoid-coated medication comprises a core composition, which is the center of the cannabinoid-coated medication, and a second composition that is disposes around the core composition.

In another aspect of this exemplary embodiment, the core composition, which is the center of the cannabinoid-coated medication, can optionally include one or more of the following: an over the counter medicine, an analgesic, a vitamin supplement.

In another aspect of this exemplary embodiment, the second composition can optionally include one or more of the following: a cannabinoid (e.g., dronabinol powder, cannabinoid resin), cannabis seed extract, hemp oil, CBD Oil or Hemp Extract or a combination thereof.

In another aspect of this embodiment, the second composition is an outer layer of the cannabinoid-coated medication covering the core composition.

The cannabinoid-coated medication comprising cannabinoid in the coating is effective for moisturizing skin.

Another advantage of the cannabinoid-coated medication comprising the amount of cannabinoid in the coating is effective for pain relief, such as for example for back pain, neck pain, shoulder pain, feet pain, wrist pain, joint pain, and/or calve pain, headache.

In yet another aspect, the cannabinoid-coated medication includes amount of cannabinoid in the coating that is an effective amount for reduction in pain associated with arthritis, back pain, backache, strains, muscle strain, sprains, bruises, muscle aches, and/or tendonitis as well as to reduce swelling, and joint stiffness.

In yet another aspect, the cannabinoid-coated medication includes amount of cannabinoid in the coating that is an effective amount for reduction dental pain.

In some aspect, the cannabinoid-coated medication includes amount of cannabinoid in the coating that is an effective amount for reduction in pain associated with menstrual cramps.

In some embodiments, the amount of cannabinoid in the cannabinoid-coated medication is from about 1% to about 20% by weight based on the total weight of the cannabinoid-coated medication.

In some embodiments, the amount of cannabinoid in the coating is from about 1% to about 20% by weight based on the total weight of the cannabinoid-coated medication.

In some embodiments, the amount of cannabinoid in the cannabinoid-coated medication is from about 1% to about 10% by weight based on the total weight of the cannabinoid-coated medication.

In some embodiments, the amount of cannabinoid in the cannabinoid-coated medication is from about 1% to about 5% by weight based on the total weight of the cannabinoid-coated medication.

In some embodiments, the amount of cannabinoid in the cannabinoid-coated medication is from about 5% to about 10% by weight based on the total weight of the cannabinoid-coated medication.

In some embodiments, the cannabinoid is cannabis sativa seed oil, In some embodiments, the cannabinoid is hemp seed extract. In some embodiments, the cannabinoid is hemp oil. In some embodiments, the cannabinoid is CBD Oil or Hemp Extract. In some embodiments, the cannabinoid is cannabinoid resin. In some embodiments, the cannabinoid is dronabinol powder.

In some embodiments, the cannabinoid is cold pressed.

In some embodiments, the cannabinoid-coated medication further comprises an effective amount of at least one analgesic.

In some embodiments, the cannabinoid-coated medication includes an amount of analgesic that is an effective amount for relieving pain, such as back pain, neck pain, shoulder pain, feet pain, wrist pain, joint pain, and/or calve pain, pain is associated with arthritis, backache, strains, muscle strain, sprains and/or bruises.

In some embodiments, the amount of analgesic in the cannabinoid-coated medication is from about 1% to about 99% by weight based on the total weight of the cannabinoid-coated medication.

In some embodiments, the amount of analgesic in the cannabinoid-coated medication is from about 1% to about 50% by weight based on the total weight of the cannabinoid-coated medication.

In some embodiments, the amount of analgesic in the cannabinoid-coated medication is from about 1% to about 40% by weight based on the total weight of the cannabinoid-coated medication.

In some embodiments, the amount of analgesic in the cannabinoid-coated medication is from about 1% to about 30% by weight based on the total weight of the cannabinoid-coated medication.

In some embodiments, the amount of analgesic in the cannabinoid-coated medication is from about 1% to about 20% by weight based on the total weight of the cannabinoid- coated medication.

In some embodiments, the amount of analgesic in the cannabinoid-coated medication is from about 5% to about 15% by weight based on the total weight of the cannabinoid- coated medication.

In one aspect of this embodiment, the cannabinoid coated on an analgesic that is trolamine salicylate.

In another aspect of this embodiment, the cannabinoid coated on an analgesic that is menthol. In some embodiments, the cannabinoid coated on an analgesic that is menthol USP.

In another embodiment, the cannabinoid-coated medication comprises additional component.

For example, the cannabinoid-coated medication comprises vitamin E. In some embodiments, the cannabinoid-coated medication comprises aloe vera. In some embodiments, the cannabinoid-coated medication comprises a concentrated blend of vitamin E and aloe vera. In some embodiments, the cannabinoid-coated medication comprises water, mineral oil, glyceryl stearate SE, cetyl alcohol, glycerin, isopropyl palmitate, sunflower (helianthus annuus) seed oil, phenoxyethanol, ethylhexylglycerin, bergamot (citrus aurantium bergamia) fruit oil, tocopheryl acetate, disodium or Ethylenediaminetetraacetic acid (EDTA), aloe vera (aloe barbadensis) leaf extract, marigold (calendula officinalis) flower extract, retinyl palmitate, and cucumber(cucumis sativus) fruit extract.

In some embodiments, the cannabinoid-coated medication further comprises neem oil. In some embodiments, the cannabinoid-coated medication further comprises Indian neem oil. In some embodiments, the cannabinoid-coated medication further comprises Karanja oil. In some embodiments, the cannabinoid-coated medication further comprises cocas nucifera (coconut) oil, hydrogenated soybean oil, ozokerite, beeswax, carthamus tinctorius (safflower) seed, oil melia azadirachta seed oil, copernicia cerifera (carnauba) wax, nigella sativa seed oil, pongamia glabra seed oil, dimethicone, melaleuca viridiflora leaf oil, tocopheryl acetate, isopropyl myristate, caprylyl glycol, phenoxyethanol, melaleuca leucadendron cajaput oil, theobroma cacao (cocoa) seed butter, butyrospermum parkii (rhea) butter, sorbitan oleate, cymbopogon schoenanthus oil, cymbopogon martini oil, citrus aurantium amara (bitter orange) peel oil, cedrus atlantica bark oil, rosmarinus officinalis (rosemary) leaf oil, melaleuca alternifolia (tea tree) leaf oil, eucalyptus globulus leaf oil, prunus amygdalus dulcis (sweet almond) oil, olea europaea (olive) fruit oil, persea gratissima (avocado) oil, helianthus annuus (sunflower) seed oil, oryza sativa (rice) bran oil, salvia sclarea (clary) oil, canarium commune gum oil and retinyl palmitate.

In some embodiments, the cannabinoid-coated medication further comprises water/aqua, ethyl hexyl stearate, butylene glycol, dimethicone, stearic acid, caprlic/capric triglyceride, glyceryl stearate, PEG-100 stearate, cetostearyl alcohol, sunflower oil, allantoin, tocopheryl acetate, ethylhexylglycerin, isohexadecane, sodium acrylate/sodium acryloyldimethyl taurate copolymer, acrylates/C10-30 alkyl acrylate crosspolymer, caprylyl glycol, hexylene glycol, polysorbate 80, disodium EDTA, D&C Green No. 5 (CI 61570), sodium hydroxide and phenoxyethanol.

In some embodiments, the cannabinoid-coated medication further comprises arctium lappa root extract, aloe barbadensis leaf extract, arnica montana flower extract, Boswellia carteril resin extract, calendula officinalis extract, camellia sinensis leaf extract, camphor, carbomer, FD&C Blue No. 1, FD&C Yellow No. 5, glycerin, Ilex paragiariensis leaf extract, isopropyl alcohol, isopropyl myristate, Melissa officinalis (lemon balm) extract, silica, tocopheryl acetate, triethanolamine, and water.

In some embodiments, the cannabinoid-coated medication is phthalate free.

In some embodiments, the cannabinoid-coated medication is paraben free.

In some embodiments, the cannabinoid-coated medication is vegan.

In some embodiments, the cannabinoid-coated medication is gluten free.

This disclosure provides a pain relief cannabinoid-coated medication comprising an effective amount of analgesic and cannabinoid.

One method of preparing the cannabinoid-coated medication involves the emulsification of a mixture containing cannabinoid followed by spray drying the mixture to form a powder. The resultant powder is readily dispersible in water and may be coated on medication containing granules, pellets, tablets and the like.

The resultant powder is readily dispersible in water and may be coated on onto the surface of medication to form a protective, enteric and/or controlled release of drug- containing granules, pellets and tablets.

### Terms

Unless otherwise defined, all technical and scientific terms used herein have the same meaning as commonly understood by a person of ordinary skill in the art to which this disclosure belongs.

As used herein, and unless stated otherwise or required otherwise by context, each of the following terms shall have the definition set forth below.

The term “a” or “an” as used herein includes the singular and the plural, unless specifically stated otherwise. Therefore, the terms “a,” “an” or “at least one” can be used interchangeably in this application.

As used herein, the term “instantaneous release layer” includes occurring or done in an instant or instantly upon the cannabinoid-coated medication ingested.

As used herein, the term “delayed release layer” includes occurring or done in delayed or later time upon being ingested. Such later time can be predetermined, for example 30 minutes after ingesting of cannabinoid-coated medication.

As used herein, the term “about” includes ±10% from the indicated values in the range. In addition, where a parameter range is provided, all integers within that range, and tenths thereof, are also provided by this disclosure. For example, “1% to 20%” includes 1.0%, 1.1%, 1.2%, 1.3%, 1.4% etc. up to 20%.

The term “analgesic” as used herein includes an analgesic medicine and can provide relief from pain.

The term “cannabinoid” as used herein can refer to a substance naturally occurring or extracted from a naturally occurring substance, or a chemically synthesized composition, compound, etc., including cannabinol and active constituents of cannabis, including extracts from hemp.

The term “cannabis seed extract” as used herein includes any extract of the cannabis plant, including extracts from marijuana and hemp. These extracts contain high levels of cannabinoids (CBD) and tetrahydrocannabinol (THC).

The term “dronabinol powder” as used herein is an orally active cannabinoid which, like other cannabinoids, has complex effects on the central nervous system, contains tetrahydrocannabinol (THC), and can provide longer-lasting pain relief and when employed in a cannabinoid-coated medication

As used herein, the term “effective amount” refers to an amount of the active component that, when contacted or sensed, is sufficient to achieve a good level of at least one desired outcome.

As used herein, the term “enteric” includes a coating that is a polymer barrier applied on medication administered orally that prevents its dissolution or disintegration in the gastric environment

As used herein, the term “Over the Counter (OTC) medicine” or “Over the Counter medicine”, is a medicine sold directly to a consumer without a prescription from a healthcare professional.

As used herein, the term “vitamin supplement”, is a product that contains one or more ingredients that are intended to supplement one's diet and are not considered food.

Each embodiment disclosed herein is contemplated as being applicable to each of the other disclosed embodiments. Thus, all combinations of the various elements described herein are within the scope of this disclosure.

### Discussion

Cannabis is a genus of plants belonging to the Cannabaceae family. The cannabis plant is widely studied and has many known beneficial properties. Species of cannabis include Cannabis sativa. Cannabis indica, and Cannabis ruderalis.

Hemp, or industrial hemp, is a strain of the Cannabis sativa plant species which is often grown specifically for industrial uses of its derived products. Hemp has, relative to cannabis, lower concentrations of tetrahydrocannabinol (THC) and higher concentrations of cannabidiol (CBD), which decreases or eliminates its psychoactive effects.

There is evidence suggesting that cannabis is safe and can be used as an inexpensive alternative to other known substances with similar properties.

The cannabinoid coated medication according to the embodiments described here provides one or more of the following benefits: enhanced absorption, reduced gastrointestinal irritation, improved gastro-resistance, enhanced efficacy of medicinal reaction, improved delayed release, and improved pain relief or reduction.

1. A coated medication comprising:
a medication; and
a coating comprising one or more cannabinoids on the exterior of the medication, in an effective amount from about 1% to about 20% by weight based on the total weight of the coated medication.

2. The coated medication of claim 1, wherein the one or more cannabinoids is hemp, or hemp oil, or cannabis seed extract, or dronabinol powder, or cannabinoid resin, or CBD oil, or hemp extract. The coated medication of claim 1, wherein the one or more cannabinoids is cold pressed hemp seed oil.

4. The coated medication of claim 1, wherein the medication comprises a medication blended with one or more cannabinoids.

5. The coated medication of claim 1, wherein the one or more cannabinoids blended with the medication is hemp, or hemp oil, or cannabis seed extract, or dronabinol, or cannabinoid resin, or CBD oil, or hemp extract.

6. The coated medication of claim 1, wherein the medication includes an analgesic, an over the counter (OTC) medication, or a vitamin supplement. The coated medication of claim 6, wherein the analgesic is selected from one or more of the following: a naproxen sodium, ibuprofen, acetaminophen, antihistamines.

8. The coated medication of claim 6, wherein the analgesic is trolamine salicylate or menthol USP.

9. The coated medication of claim 6, wherein the vitamin supplement is selected from one or more of the following: a multivitamin, a dietary supplement with vitamins, dietary minerals, or other nutritional elements.

10. The coated medication of claim 1, wherein the coating has at least one instantaneous release layer and one delayed release layer.

11. The coated medication of claim 10, wherein the at least one delayed release layer comprises an enteric coating.

12. The coated medication of claim 1, wherein the medication comprises a core composition, which is the center of the medication, and a second composition that is disposed around the core composition.

13. The coated medication of claim 12, wherein the core composition further comprises an over the counter (OTC) medication, an analgesic, or a vitamin supplement.

14. The coated medication of claim 6, wherein the amount of analgesic in the medication is from about 1% to about 99% by weight based on the total weight of the medication.

15. The coated medication of claim 6, further comprising a concentrated blend of vitamin E and aloe vera.

16. The coated medication of claim 1, wherein the coated medication is in the form of a tablet, capsule, gel capsule, liquid capsule, gummy bear, caplet, orally disintegrating tablet, pill, lozenge, pastille or pellet.

17. The coated medication of claim 1, wherein the medication is phthalate free, paraben free, vegan, and gluten free.

18. A method for preparing a cannabinoid-coated medication comprising:
a. emulsifying a mixture containing cannabinoid;
b. spray drying the mixture to form a powder;
c. dispersing the powder n water;
d. using the water to coat the medication.
